(19) United States
(12) Patent Application Publication
(10) Pub. No.: US 2022/0168470 A1
(43) Pub. Date: Jun. 2, 2022

(54) MATERIAL AND SYSTEM FOR THE THERAPEUTIC TREATMENT OF JOINTS

(71) Applicants: VIMEX SPOLKA Z OGRANICZONA ODPOWIEDZIALNOSCIA (VIMEX), Gliwice (PL); H&D WIRELESS AB; PLASMACHEM PRODUKTIONS UND HANDEL GMBH, Berlin (DE); IMAGE GUIDED THERAPY SA, Pessac (FR); ISTITUTO ORTOPEDICO RIZZOLI, Bologna (IT); BAR ILAN UNIVERSITY, Ramat Gan (IL); SCUOLA SUPERIORE DI STUDI UNIVERSITARI E DI PERFEZIONAMENTO SANT'ANNA, Pisa (IT)

(72) Inventors: Leonardo RICOTTI, Peccioli (IT); Lorenzo VANNOZZI, San Miniato (IT); Andrea CAFARELLI, Carrara (IT); Gilbert Daniel NESSIM, Ramat Gan (IL); Gina LISIGNOLI, Bologna (IT); Aharon WECHSLER, Shoham (IL); Erik Jean-Claude DUMONT, Pessac (FR); Carsten JOST, Berlin (DE); Tomasz GAPINSKI, Zabrze (PL); Pär BERGSTEN, Solna (SE); Elena GABUSI, Bologna (IT); Milena FINI, Bologna (IT); Matilde TSCHON, Bologna (IT); Alessandro RUSSO, Bologna (IT); Stefano ZAFFAGNINI, Bologna (IT); Riccardo MELICONI, Bologna (IT); Yirij FEDUTIK, Berlin (DE); Krzysztof Stanislaw LENARTOWICZ, Rybnik (PL); Åke JERNBERGER, Sollentuna (SE); Yonatan SHACHAF, Haifa (IL); Magnus ERIKSSON, Tyresö (SE)

(73) Assignees: VIMEX SPOLKA Z OGRANICZONA ODPOWIEDZIALNOSCIA (VIMEX), Gliwice (PL); H&D WIRELESS AB, Kista (SE); PLASMACHEM PRODUKTIONS UND HANDEL GMBH, Berlin (DE); IMAGE GUIDED THERAPY SA, Pessac (FR); ISTITUTO ORTOPEDICO RIZZOLI, Bologna (IT); BAR ILAN UNIVERSITY, Ramat Gan (IL); SCUOLA SUPERIORE DI STUDI UNIVERSITARI E DI PERFEZIONAMENTO SANT'ANNA, Pisa (IT)

(21) Appl. No.: 17/433,470

(22) PCT Filed: Feb. 25, 2020
(86) PCT No.: PCT/IB2020/051603

§ 371 Date: Aug. 24, 2021

(30) Foreign Application Priority Data
Feb. 25, 2019 (IT) 102019000002697

(51) Int. Cl. A61L 27/38; A61L 27/44; A61L 27/52; A61M 37/00; A61N 1/20; A61N 1/32; B82Y 5/00

(52) U.S. Cl. CPC A61L 27/3834; A61L 27/3852; A61L 27/443; A61L 27/446; A61L 27/52; A61N 2007/0004; A61N 1/205; A61N 1/326; B82Y 5/00; A61L 2400/06; A61L 2400/12; A61M 37/0092

(57) ABSTRACT

A system for the therapeutic treatment of joints comprising a composite material comprising a biodegradable polymer matrix and a plurality of piezoelectric particles adapted to generate local electric charges in response to an external stimulation made by means of ultrasound, said plurality of piezoelectric particles being dispersed in the matrix. The composite material also comprises a plurality of stamina cells dispersed in the biodegradable polymer matrix and a plurality of carbon-based particles. The system also comprises a releasing device, arranged to deposit the composite material in a joint cavity at predetermined areas of the cartilage, and a stimulator device arranged to emit ultrasound at a predetermined frequency, a predetermined intensity and for a predetermined time of application, in such a way that, when the device is located near a joint wherein the composite material has been deposited, said ultrasound stimulate the plurality of piezoelectric particles.

### FIELD OF THE INVENTION

The present invention relates to the therapeutic treatment of joints.

In particular, the invention relates to a composite material and a treatment system for the regeneration of cartilage tissue.

### DESCRIPTION OF THE PRIOR ART

As known, osteoarthritis is currently the most common rheumatological disease: it causes severe motor disabilities and pain, which prevent regular daily life activities. This pathology mostly affects the elderly or obese population, although the cases found in sportsmen of young age following joint injuries are not negligible.

Osteoarthritis is a chronic degenerative disease and is manifested by symptoms that are difficult to diagnose in the initial stages, but which intensify quickly over time. If left untreated in the appropriate time and manner, the level of disability can gradually increase over time. At the level of the joint, due to this pathology, lesions on the cartilaginous surface and at the osteo-chondral interface can occur. The aggravation of the conditions can quickly lead to the final stage, which involves the implantation of a joint prosthesis.

In physiological conditions, the articular cartilage is responsible for the correct functioning of the joints, distributing the load (function of “shock absorber”) and guaranteeing the necessary lubrication to avoid further problems from rubbing between tissues. Cartilage alterations can cause joint pain, given by the inability of the joint to support the load adequately.

In these early symptomatic phases, a preservation therapy of the joint structure is necessary. The great majority of this type of therapy is based on viscosupplements or platelet enriched plasma (PRP), which are able to promote a lubricating action or an anti-inflammatory effect on the compromised joint, thus ensuring a better patient condition.

However, both viscosupplements and PRP must be injected periodically into the joint, given that this type of treatment relieves the patient's condition only in the short term. The beneficial effects of these treatments are also very limited and highly dependent on the patient.

Once the degeneration of the cartilage tissue reaches a level of degeneration that can no longer be treated with the therapies mentioned above, osteoarthritis is treated surgically. Common practices are microfracture, autologous chondrocyte transplantation, autologous osteochondral transplantation and synthetic scaffolds.

However, even these systems appear to lose their effectiveness over time, often requiring an absolutely much more invasive intervention, such as the implantation of a knee prosthesis.

In addition, both the injections and the transplants mentioned above can lead to inflammations that are difficult to cure.

A further disadvantage of these therapies concerns the very high costs that the patient has to bear, especially in case of poor efficacy of the treatment, for which frequent reiteration is necessary.

Recently, some scientific studies have considered ultrasonic stimulation of piezoelectric nanomaterials for cell regeneration, in particular neuronal and muscle cells.

In US2012121712, for example, a method is described which provides for the induction of a non-invasive stimulation of neuronal cells, both in vitro and in vivo, through the use of piezoelectric nanovectors. Specifically, these are boron nitride nanotubes (BNNT) capable of being internalized by the cells and of converting a specific non-invasive external stimulus (ultrasound) into electrical inputs capable of stimulating the cells themselves. The nanotransducers are optionally coated with specific polymers. This method aims to reduce the high invasiveness typical of electrical cell stimulation (electrotherapy) methodologies that make use of electrodes to be inserted near the cellular tissues to be stimulated.

In addition, in the state of the art there are studies that take into consideration the ultrasonic stimulation of piezoelectric nanomaterials in order to treat and regenerate articular cartilage. Some examples are reported in “Piezoelectric smart biomaterials for bone and cartilage tissue engineering” by Jaicy Jacob et al. (https://inflammregen.biomedcentral.com/articles/10.1186/s41232-018-0059-8) and in “Current Trends in Fabrication of Biomaterials for Bone and Cartilage Regeneration: Materials Modifications and Biophysical Stimulations” by Agata Przekora (https://www.mdpi.com/1422-0067/20/2/435).

However, the composite materials used in the state of the art do not optimize the distribution of the electric charges generated by the piezoelectric particles in the entire volume of the polymer matrix, making the cell regeneration process less effective and homogeneous, and typically do not have mechanical properties and optimal friction for cartilage applications.

### SUMMARY OF THE INVENTION

It is therefore a feature of the present invention to provide a system comprising a composite material for the therapeutic treatment of joints that allows to significantly slow down or even reverse the degenerative and inflammatory process affecting the articular cartilage, slowing down or avoiding the need to resort to a joint prosthesis.

It is also a feature of the present invention to provide such a system for avoiding the drawbacks, in terms of costs and efficiency, of articular therapies of the prior art.

It is also a feature of the present invention to provide such a system that exploits the known principle of ultrasonic stimulation of piezoelectric nanomaterials for the regeneration of cartilage tissues.

It is also a feature of the present invention to provide a method for the therapeutic treatment of joints which involves the use of this system and of this composite material.

These and other objects are achieved by a composite material arranged to treatment therapeutic of joints comprising:
a biodegradable polymer matrix;
a plurality of piezoelectric particles adapted to generate local electric charges in response to an external stimulation made by means of ultrasound, said plurality of piezoelectric particles being dispersed matrix;
a plurality of stamina cells dispersed in the biodegradable polymer matrix;
   whose main feature is that in the biodegradable polymer matrix are also dispersed carbon-based particles.

According to another aspect of the invention, a system for the therapeutic treatment of joints is also claimed comprising:
a composite material comprising:
   a biodegradable polymer matrix;
   a plurality of piezoelectric particles adapted to generate local electric charges in response to an external stimulation made by means of ultrasound, said plurality of piezoelectric particles being dispersed in the matrix;
   a plurality of stamina cells dispersed matrix;
a releasing device arranged to deposit the composite material in a joint cavity at predetermined areas of the cartilage;
a stimulator device arranged to emit ultrasound at a predetermined frequency, a predetermined intensity and for a predetermined time of application, in such a way that, when the device is located near a articulation wherein said composite material has been deposited, the ultrasound stimulate the plurality of piezoelectric particles;
   whose main feature is that in the biodegradable polymer matrix are also dispersed carbon-based particles.

When the composite material is stimulated by means of ultrasound, the piezoelectric particles generate electric charge that have an chondrogenic effect on the stamina cells, going to regenerate the cartilage and possibly having an anti-inflammatory effect.

In particular, the carbon-based particles allow to increase the mechanical resistance and to decrease the friction coefficient of the composite material, in addition to allowing a more effective distribution, within the matrix, of the electric charges generated by the piezoelectric particles.

In particular, the stamina cells are selected from the group consisting of:
autologous stamina cells;
heterologous stamina cells;
a combining the previous.

In particular, the piezoelectric particles are nanoparticles.

Advantageously, piezoelectric nanoparticles can have the form of “nanotubes”, “nanowires”, “nanorods”, “nanospheres”, “nanobelts”, “nanowalls”, “nanodisks”, “nanoplates”, “nanotripods”, or others.

In particular, the piezoelectric nanoparticles are selected from the group consisting of:
barium titanate particles;
zinc oxide particles;
piezoelectric polymer particles, such as PVDF or P (VDF-TrFE);
KNN or NKN particles;
alkaline niobate particles;
PZT particles;
boron nitride particles;
PM PMN-PT particles;
a combining the previous.

In particular, the biodegradable polymer matrix has viscosity set between 10 mPa*s and 10⁵ mPa*s.

In particular, the piezoelectric particles are previously subject to chemical functionalization of the surface and/or coating with chemical groups which facilitate the inclusion and dispersion of these particles in the polymer matrix and/or which improve their biocompatibility.

In particular, said biodegradable polymer matrix is a hydrogel.

The use of a composite material in the form of gel allows the deposit and the maintenance of piezoelectric particles and stamina cells within defects that can be present on the surface of the cartilaginous tissue, remarkably increasing the efficiency of the treatment with respect to the prior art.

In particular, the carbon-based particles can be carbon nanotubes, or graphene, graphene oxide, reduced graphene oxide or other in the form of a single layer, laminar structures or other. These particles can also be chemically functionalized.

In particular, chondrocytes are also dispersed in the biodegradable polymer matrix.

Alternatively, other cells with paracrine action can be dispersed in the matrix with respect to the stamina cells.

In particular, the matrix comprises biomolecules arranged for inducing the stamina cells to evolve into chondrocytes.

In particular, the composite material is deposited at degradations and/or degenerations and/or defects of the cartilage.

Advantageously, a wearable device is also comprised comprising said stimulator device, said wearable device being configured to place the stimulator device near a joint.

Advantageously, a monitoring apparatus is also comprised arranged for monitoring the regeneration status of the cartilaginous tissue.

In particular, the monitoring apparatus comprises a device for ultrasound scanning.

Advantageously, a control unit is also comprised configured for:
receiving data that correlate values of parameters characterizing the ultrasound with the speed and the quality of the cartilaginous tissue regeneration;
setting the characterizing parameters on ranges of values that guarantee a greater speed and quality of the cartilaginous tissue regeneration.

In particular, the characterizing parameters are selected from the group consisting of:
frequency of said ultrasound;
intensity of said ultrasound;
time of applying said ultrasound;
duty cycle of said ultrasound;
a combining the previous.

According to a further aspect of the invention, a method for the therapeutic treatment of joints is also claimed comprising the steps of:
prearranging a composite material comprising:
   a biodegradable polymer matrix;
   a plurality of piezoelectric particles adapted to generate local electric charges in response to an external stimulation made by means of ultrasound, said plurality of piezoelectric particles being dispersed in the matrix;
   a plurality of stamina cells dispersed in the matrix;
depositing the composite material in a joint cavity at predetermined areas of the cartilage;
transmitting ultrasound at said joint cavity for stimulating the plurality of piezoelectric particles.

Advantageously, a step is also provided of application of a wearable device at the articulation, said wearable device comprising at least one ultrasound emitter.

In particular, a step is also provided of monitoring the articulation for monitoring the regeneration status of the cartilaginous tissue.

In particular, the step of monitoring is made by means of ultrasound scanning.

Advantageously, are also provided the steps of:
collecting data that correlate values of parameters characterizing the ultrasound with the speed and the quality of the cartilaginous tissue regeneration;
setting the characterizing parameters on ranges of values that guarantee a greater speed and quality of the cartilaginous tissue regeneration.

In particular, the characterizing parameters are selected from the group consisting of:
frequency of said ultrasound;
intensity of said ultrasound;
time of applying said ultrasound;
duty cycle of said ultrasound;
a combining the previous.

### BRIEF DESCRIPTION OF THE DRAWINGS

Further characteristic and/or advantages of the present invention are more bright with the following description of an exemplary embodiment thereof, exemplifying but not limitative, with reference to the attached drawings in which:

FIG. 1 shows schematically the composite material according to the present invention;

FIG. 2 shows the deposit of the composite material in the joint by the releasing device;

FIG. 3 shows the wearable device arranged on the leg of a patient and a stimulator device adapted to emit ultrasound;

FIG. 4 shows a possible flow-sheet of the operations made by the system according to the present invention;

FIG. 5 shows a variant of the flow-sheet of FIG. 4, wherein a step of monitoring is further provided.

### DESCRIPTION OF A PREFERRED EXEMPLARY EMBODIMENT

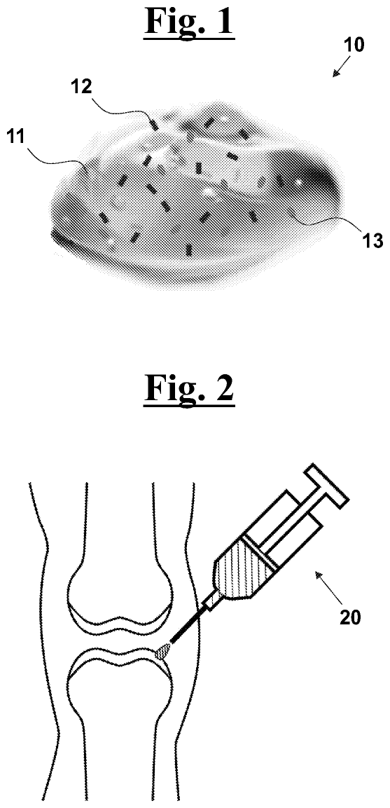

FIG. 1 shows an exemplary embodiment of the composite material 10, according to the present invention, comprising a biodegradable polymer matrix 11 in which a plurality of piezoelectric particles 12 and a plurality of stamina cells 13 are dispersed, in addition to a plurality of carbon-based particles.

In particular, the carbon-based particles allow to increase the mechanical resistance and to decrease the friction coefficient of the composite material, in addition to allowing a more effective distribution, within the matrix, of the electric charges generated by the piezoelectric particles.

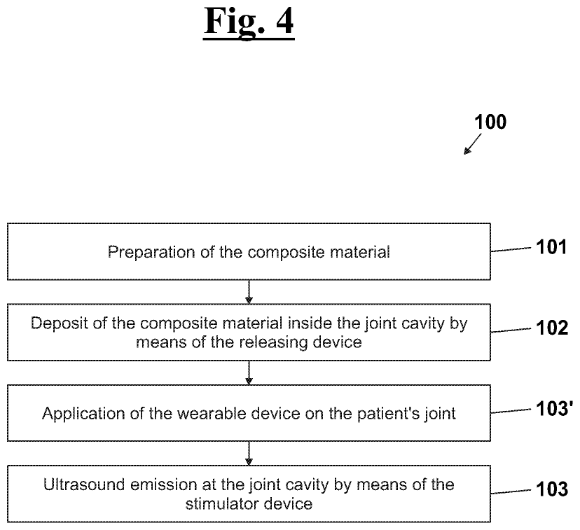

FIG. 4 shows a possible flow chart 100 of the operations made by the system according to the present invention.

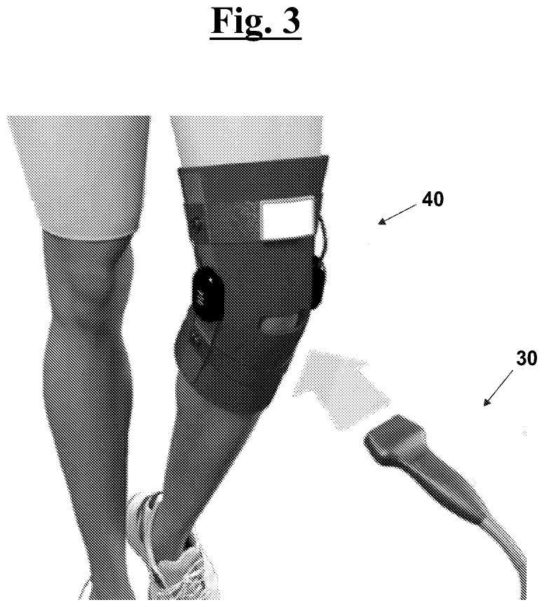

In particular, with reference even at FIGS. 2 and 3, after having prepared the composite material 10 [101], the composite material 10 is adapted to be deposited by a releasing device 20, for example by injection, in a joint cavity at degradations and/or at defects of the cartilage of the articulation to treat [102].

When the composite material 10 is stimulated by means of ultrasound emitted by the stimulator device 30 provided by the present invention, the piezoelectric particles are stimulated and, consequently, generate electric charges which have a chondrogenic on the stamina cells, going to regenerate the cartilage [103].

The system can furthermore provide a wearable device 40 that can contain the stimulator device 30 inside, in order to direct the ultrasound towards the joint cavity and stimulate the piezoelectric particles automatically at predetermined intervals [103′].

In the preferred exemplary embodiment of FIG. 1, the biodegradable polymer matrix 11 is, in particular, a hydrogel. This allows the material 10 to have enough density and viscosity for keeping the piezoelectric particles 12 and the stamina cells 13 at close distance so as to increase their interaction. Furthermore, the form of hydrogel allows the composite material 10 to be injected in the joint by adequately filling the joint cavity and coming into contact with each lesion of the cartilage, then remaining in a stable position.

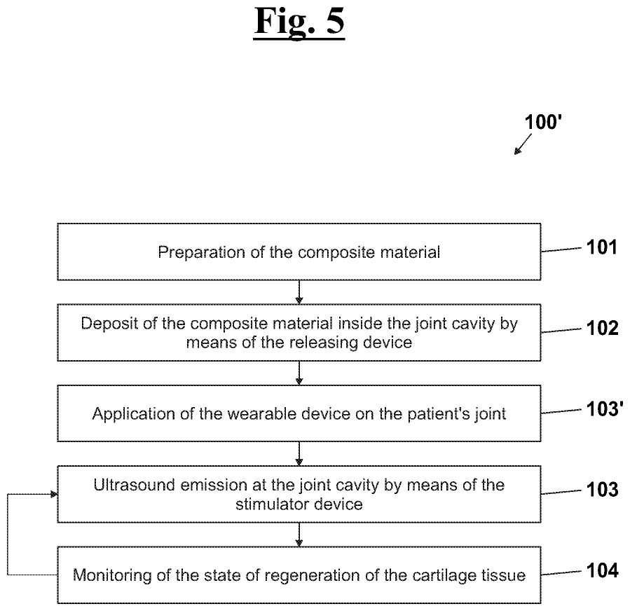

FIG. 5 shows a variant of the flow-sheet of FIG. 4, wherein a step of monitoring the regeneration status of the cartilaginous tissue is further provided, operated by a control unit. On the basis of this monitoring, the control unit can then subsequently modify the characterizing parameters of the ultrasounds in order to guarantee greater speed and quality of the cartilage tissue regeneration.

1. A system for the therapeutic treatment of joints comprising:
a composite material comprising:
a biodegradable polymer matrix;
a plurality of piezoelectric particles adapted to generate local electric charges in response to an external stimulation made by means of ultrasound, said plurality of piezoelectric particles being dispersed in said biodegradable polymer matrix;
a plurality of stamina cells dispersed in said biodegradable polymer matrix;
a releasing device arranged to deposit said composite material in a joint cavity at predetermined areas of the cartilage;
a stimulator device arranged to emit ultrasound at a predetermined frequency, a predetermined intensity and for a predetermined time of application, in such a way that, when said device is located near a articulation wherein said composite material has been deposited, said ultrasound stimulate said plurality of piezoelectric particles;
said system characterized in that in said biodegradable polymer matrix are also dispersed carbon-based particles.

2. The system for the therapeutic treatment of articulations, according to claim 1, wherein a wearable device is also provided comprising said stimulator device, said wearable device being configured for arranging said stimulator device near a joint.

3. The system for the therapeutic treatment of articulations, according to claim 1, wherein a monitoring apparatus is also comprised arranged for monitoring the regeneration status of the cartilaginous tissue.

4. The system for the therapeutic treatment of articulations, according to claim 3, where a control unit is also comprised configured for:
collecting data that correlate values of parameters characterizing said ultrasound with speed and quality of the cartilaginous tissue regeneration;
setting said characterizing parameters on ranges of values that guarantee greater speed and quality of the cartilaginous tissue regeneration.

5. The system for the therapeutic treatment of articulations, according to claim 4, wherein said characterizing parameters are selected from the group consisting of:
frequency of said ultrasound;
intensity of said ultrasound;
time of applying said ultrasound;
duty cycle of said ultrasound;
a combining the previous.

6. A composite material arranged to the therapeutic treatment of joints, said composite material comprising:
a biodegradable polymer matrix;
a plurality of piezoelectric particles adapted to generate local electric charges in response to an external stimulation made by means of ultrasound, said plurality of piezoelectric particles being dispersed in said biodegradable polymer matrix;
a plurality of stamina cells dispersed in said biodegradable polymer matrix;
said composite material characterized in that in said biodegradable polymer matrix are also dispersed carbon-based particles.

7. The composite material, according to claim 6, wherein said biodegradable polymer matrix is a hydrogel.

8. The composite material, according to claim 6, wherein in said biodegradable polymer matrix are also dispersed chondrocytes.

9. The composite material, according to claim 6, wherein said biodegradable polymer matrix comprises biomolecules configured for inducing said stamina cells to evolve into chondrocytes.
